(19) United States
(12) Patent Application Publication
(10) Pub. No.: US 2005/0181066 A1
(43) Pub. Date: Aug. 18, 2005

(54) Novel use of guanylate cyclase activators for the treatment of respiratory insufficiency

(75) Inventors: Friedrich Josef Grimminger, Butzbach (DE); Ralph Schermuly, Mengerskirchen (DE); Christian Schudt, Konstanz (DE)

Correspondence Address: NATH & ASSOCIATES PLLC, 1030 FIFTEENTH STREET, N.W., SIXTH FLOOR, WASHINGTON, DC, 20005, US

(73) Assignee: Altana Pharma AG, Konstanz (DE)

(21) Appl. No.: 10/512,547

(22) PCT Filed: Apr. 24, 2003
(86) PCT No.: PCT/EP03/04243

(30) Foreign Application Priority Data
Apr. 26, 2002 (EP) 02009552.7

(51) Int. Cl. A61K 38/10; A61K 31/538; A61K 31/5377; A61K 31/522; A61K 31/519

(52) U.S. Cl. 424/608; 514/234.5; 514/252.16; 514/260.1; 514/15; 514/45; 514/263.34; 514/509; 514/303; 514/406; 514/230.5; 514/248; 514/602

(57) ABSTRACT

The invention relates to the novel use of guanylate cyclase activators for the treatment of partial and global respiratory failure.

### TECHNICAL FIELD

The invention relates to novel use of guanylate cyclase activators in the treatment of pulmonary disorders.

### PRIOR ART

In the healthy lung both at rest and during exercise there are always areas of good and poor or absolutely no ventilation existing simultaneously side by side (ventilation inhomogeneity). An as yet unknown mechanism ensures that there is little or no perfusion of the capillaries adjacent to alveoli with little or no ventilation. This occurs in order to minimize inefficient perfusion of areas of the lung which are not involved in gas exchange.

Necessary for efficient gas exchange in the lung is a dynamic adaptation of the perfusion conditions to the continual changes in regional ventilation. This coupling is referred to as matching and is determined qualitatively and quantitatively as the V/Q (V=ventilation; Q=perfusion) ratio by means of the multiple inert gas elimination technique (MIGET).

During bodily exercise, the distribution of ventilation changes (recruitment of new alveoli) and there is increased perfusion of the relevant capillary bed. Conversely, when there is less ventilation due to physiological or pathological processes (airway obstruction), the capillary flow are reduced through vasoconstriction. This process is referred to as “hypoxic vasoconstriction” (Euler-Liljestrand mechanism).

When this adaptation mechanism is impaired (“mismatch”), there may, despite adequate ventilation and normal perfusion of the lungs, be a more or less pronounced collapse of the gas exchange function, which can be compensated only inadequately despite a further increase in ventilation or perfusion. Under these conditions there are regions which are not ventilated but are well perfused (shunting) and those which are well ventilated but not perfused (dead space ventilation), and all intermediate states characterized by deviations from the normal value of V/Q=1. These are, on the one hand, low-V/Q areas (hyperperfusion with little ventilation), and on the other hand high-V/Q areas (hypoperfusion with hyperventilation). The consequences of this mismatch are hypoxaemia (deterioration in gas exchange with decrease in the oxygen content of the patient's blood), wasted perfusion (uneconomical perfusion of unventilated areas) and wasted ventilation (uneconomical ventilation of poorly perfused areas). This leads to a limitation in the patient's performance due to a deficient oxygen supply to the muscles in combination with a “squandering” of cardiorespiratory reserves. The clinical symptoms are a limitation on performance and exercise-dependent or permanent dyspnoea.

In patients with inflammatory and degenerative lung disorders such as, for example, chronic obstructive bronchitis (COPD), bronchial asthma, pulmonary fibroses, emphysema, interstitial pulmonary disorders and pneumonias there is observed to be partial or global respiratory failure. The cause is inadequate adaptation of the intrapulmonary perfusion conditions to the inhomogeneous pattern of the distribution of ventilation. The mismatch derives from the effect of vasoactive (inflammatory) mediators which prevail over the physiological adaptation mechanism. This effect is particularly evident during exercise and when the oxygen demand is increased and it is manifested by dyspnoea (hypoxia) and limitation of performance.

Administration of vasodilators (endothelin antagonists, angiotensin II antagonists, prostacyclin [systemically administered, orally or intravenously], calcium channel blockers) may considerably exacerbate the impairment of the gas exchange function, caused by nonselective vasodilation, especially in the poorly ventilated areas of the lungs, resulting in an increase in mismatch and shunting.

Administration of a vasodilator (especially nitric oxide, NO) by inhalation has a theoretically preferred effect only in the well-ventilated areas of the lungs. However, this requires an efficient inhalation technique which is troublesome for the patient. Additional factors are the systemic effects on absorption through the alveolar epithelium (especially with substances having a long duration of action) and the possible irritation of the bronchial system.

Bronchodilators are intended to reduce airway obstruction which is present. However, in previously damaged lungs they may in fact aggravate further the mismatch, which is the main cause of the reduced performance, through increasing the ventilation in so-called high-V/Q areas and by unwanted systemical vasodilatation (increase in perfusion in low-V/Q areas).

Thus, Maurenbrecher H et al. [Maurenbrecher H et al. (2001) Chest 120: 573] describe experiments on improving oxygenation through administration of NO by inhalation in an ARDS (acute respiratory distress syndrome) pig model. In the introduction, the authors describe the known mode of action of endogenous and exogenous NO on the activity of guanylate cyclase and thus on the generation of GTP (cyclic guanosine 3′/5′-triphosphate). The authors emphasize that vasodilators administered as infusion, such as an NO donor or a prostaglandin, reduce pulmonary hypertension (PHT) but at the same time worsen the arterial oxygenation, since these substances increase the blood flow in the unventilated regions and thus have an unwanted hypotensive systemic effect. Accordingly, a gas exchange impairment is described as being induced in parallel with the vasorelaxation on systemic administration of vasodilators such as NO donors and prostaglandins, and is attributable to a ventilation/perfusion mismatch. The authors do not describe the effect of nonselective vasodilators such as, for example, guanylate cyclase activators.

Walmrath D et al. [Walmrath D et al. (1997) Eur. Respir. J. 10: 1084] describe the selective vasodilating effect of inhaled NO and the selective pulmonary vasodilatation caused thereby, and the improvement, associated therewith, in gas exchange in the lung. The effects of inhaled versus systemic prostanoids and those of inhaled nitric oxide on gas exchange in an isolated perfused rabbit lung model are described. The authors report that these substances have pulmonary vasoactivity on inhalation or else infusion. However, according to the authors, systemic administration (infusion) of these substances leads to a deterioration In gas exchange (mismatch); this effect does not occur on administration of these substances by inhalation. The authors emphasize that the mechanism of the selective mode of action of inhaled vasodilators is based on deposition in well ventilated areas of the lung. The authors do not describe the effect of systemically administered nonselective vasodilators such as, for example, guanylate cyclase activators.

Rossaint R et al. [Rossaint R et al. (1993) New England Journal of Medicine 328: 399] describe the effect of inhaled nitric oxide (NO) on the intrapulmonary blood flow and the gas exchange in ventilated patients suffering from acute adult respiratory distress syndrome (ARDS). It is explained that the documented improvement in gas exchange through inhaled NO is based on selective vasodilatation in well ventilated regions of the lung and thus leads to an improvement in the adaptation of ventilation and perfusion. The authors do not describe the effect of systemically administered nonselective vasodilators such as, for example, guanylate cyclase activators. On the contrary, this article suggests to the skilled person that only administration of a vasodilator by inhalation, and the preferential deposition, associated therewith, of the vasodilator in well ventilated areas of the lung, leads to relaxation of the vessels preferentially in these regions of the lung and thus to an improvement in the ventilation/perfusion matching and the gas exchange.

Didrik S O [Didrik Saugstad O (1999) Lancet 354: 1047] describes the role of inhaled nitric oxide (NO) as medicament for the treatment of persistent pulmonary hypertension in neonates. The author explains the known intracellular signal pathway of NO and the effect of inhaled NO resulting therefrom. Stranak Z et al. [Stranak Z et al. (1996) Eur. J. Pediatr. 155: 907] describe the effects of inhaled nitric oxide (NO) on the alveolar-arterial oxygen difference (AaDO2) and on the oxygenation index (OI) in 15 neonates with severe respiratory insufficiency. The study results suggest that an improvement in gas exchange is possible on treatment of neonates with inhaled NO. The authors do not describe the effect of systemically administered nonselective vasodilators such as, for example, guanylate cyclase activators.

In a further study, Annest S J et al. [Annest S J et al. (1981) The Journal of Trauma 21: 1029] describe the use of sodium nitroprusside (Np) and nitroglycerin (Ng), both substances which lead to release of nitric oxide in the body, on 11 patients with post-traumatic pulmonary dysfunction. The authors state that infusion of Np and Ng leads to a reduction in the acute pulmonary hypertension and is associated with a deterioration in the arterial oxygen content. The authors conclude from this that the pulmonary hypertension associated with post-traumatic acute pulmonary dysfunction must have been produced by vasoconstriction in regions of the lung with poor or absolutely no ventilation. They postulate that this is caused by so-called hypoxic vasoconstriction. Thus, infusion of the vasodilators Np and Ng leads via relaxation of the constricted vessels to a reduction in the pulmonary pressure, but with the consequence of a deterioration in gas exchange through admixture of low oxygen-saturated blood. The authors do not describe the effect of systemically administered nonselective vasodilators such as, for example, guanylate cyclase activators. On the contrary, this article suggests to the skilled person that a mismatch is to be expected on systemic administration of a vasodilator.

Bencowitz H Z et al. [Bencowitz H Z et al. (1984) Journal of the American College of Cardiology 4: 918] describe the effect of sodium nitroprusside (Np) on the gas exchange function and the systemic circulatory function in 5 patients with impairments of the pumping function of the left heart (congestive heart failure) and with impairments of the gas exchange function (respiratory failure). According to the authors, infusion of Np increases the ventilation-perfusion mismatch in the lung. The authors emphasize that although infusion of Np has a beneficial effect on heart function (cardiac output) and oxygen transport in the studied patients, Np has an adverse effect on the ratio of ventilation and perfusion in the lung and possibly has harmful effects for patients with heart failure.

A paper by Schermuly R T et al. [Schermuly R T et al. (2001) Am J. Respir. Cell. Mol. Biol. 25: 219] describes the effect of urodilatin—a natriuretic peptide which activates particulate guanylate cyclase—and dipyridamole—a phosphodiesterase 5 inhibitor—in a rabbit model of acute pulmonary hypertension. According to the authors, systemic administration both of urodilatin and of dipyridamole led to a dose-dependent reduction in the pulmonary and systemic vasodilatation. A dose of dipyrimadole which shows no activity per se leads to a marked enhancement of the vasodilatation caused by urodilatin in the animal model described. The results of the study show that intravenous administration of the guanylate cyclase activator urodilatin leads to a marked reduction in pulmonary hypertension but also reduces the pressure in the systemic circulation to the same extent and thus has no selectivity in relation to the pulmonary circulation. The statements by Schermuly et al. do not relate to the effects on gas exchange due to administration of the vasodilators. The authors describe the effect of the vasodilators on the systemic and pulmonary haemodynamics; no effect of the vasodilators on gas exchange is described. Accordingly, it is evident to the skilled person that the guanylate cyclase activator used has no selectivity for the pulmonary circulation and is therefore unsuitable as substance for treating respiratory failure.

Forssmann W et al. [Forssmann W et al. (2001) Cardiovascular Research 51: 458] also describe the use of urodilatin; in this case as possible use for the treatment of bronchoconstriction and acute asthma. The authors' arguments are based on studies in which intravenous administration of urodilatin led to an improvement in the ventilatory parameters in patients with bronchial asthma. The authors base the bronchodilating effect of urodilatin on the increase in the intracellular cGMP levels in bronchial smooth muscle cells. The essential content of the paper by Forssmann W et al. is thus the improvement in the ventilatory restrictions of patients with asthma owing to the effect of urodilatin on bronchial smooth muscle cells. The authors thus describe the effect of a guanylate cyclase activator on the ventilatory efficiency (reduction in airway resistance). The oxygen content of the blood and thus aspects of ventilation/perfusion matching (gas exchange) are not described by Forssmann W et al. Hence, according to Forssmann W et al., a systemically administered bronchodilator is expected however to result in a deterioration in matching via a nonselective vasodilatation in poorly ventilated areas of the lung, despite an improvement in overall ventilation.

Examples III and VI of WO 9009171 describe various combinations of a guanylate cyclase activator with bronchodilators. Example III describes the use of guanylate cyclase activator with isosorbide dinitrate (a beta-blocker with a potentially bronchoconstricting effect) and an antiarrhythmic. Example VI describes the guanylate cyclase activator isosorbide mononitrate with amiodarone (an antiarrhythmic) and likewise with a beta-blocker.

A whole series of guanylate cyclase activators are known from the prior art and are described as substances for the treatment of asthma, diabetes, stroke or pulmonary hypertension.

### DESCRIPTION OF THE INVENTION

The object of the present invention is thus to provide a substance which, on oral, intravenous or else inhalational administration, leads on the one hand to the preferred dilatation of vessels in the pulmonary circulation (pulmonary selectivity) and, at the same time, to a redistribution of the blood flow within the lung in favour of the well-ventilated areas (intrapulmonary selectivity).

It has now been found, surprisingly, that guanylate cyclase activitators are suitable for the treatment of patients having the abovementioned mismatch. Administration of guanylate cyclase activitators leads to dilatation of vessels in the pulmonary circulation and, at the same time, to a redistribution of the blood flow within the lung in favour of the well-ventilated areas. This principle, referred to hereinafter as rematching, leads to an improvement in the gas exchange function both at rest and during physical exercise.

Contrary to the skilled person's expectation, that the vasodilating effect achieved with a guanylate cyclase activitator has neither pulmonary or intrapulmonary selectivity, it emerges that there was not only no deterioration but in most cases a significant improvement of pre-existent gas exchange impairments In the treated patients. Guanylate cyclase activitators are thus suitable as rematching medicament. This improvement in the oxygen supply is not brought about by the well-known general (pulmonary and systemic) vasorelaxation which is typical of guanylate cyclase activitators. On the contrary, the improvement in gas exchange derives from guanylate cyclase activitators bringing about or enhancing a lung-selective and intrapulmonary-selective vasodilatation in the well-ventilated regions. It is thus possible in patients with a pronounced gas exchange impairment to improve markedly a restricted oxygen supply through administration of guanylate cyclase activitators. In addition, the functional capacity of these patients is significantly improved through a reduction in the ventilation of unperfused areas of the lung (wasted ventilation) and the perfusion of unventilated areas of the lung (wasted perfusion).

The invention thus relates to the use of guanylate cyclase activators for producing medicaments for the treatment of partial and global respiratory failure. This use is preferably for patients who have a mismatch of pulmonary ventilation and pulmonary perfusion.

The mechanism of the intrapulmonary-selective effect of guanylate cyclase activators is based on the inhomogeneity of substrate distribution (cGMP) caused by vasodilatation during normal ventilation.

According to this invention, respiratory failure relates to an impairment of oxygen uptake or carbon dioxide release in the lung. Partial respiratory failure according to the invention relates to a fall in the O₂ partial pressure in the blood (PaO₂<60 mmHg) as a manifestation of the aforementioned impairment of oxygen uptake or carbon dioxide release. According to this invention, global respiratory failure relates to a fall in the O₂ partial pressure in the blood and a rise in the CO₂ partial pressure in the blood (PaO₂<60 mmHg, PaCO₂>50 mmHg) as a manifestation of the aforementioned impairment of oxygen uptake or carbon dioxide release.

According to this invention, vasodilatation during normal ventilation relates to a local increase in activity of NO synthase in well-ventilated lung areas due to alveolar distension. This results in an increased cGMP synthesis (activation of guanylate cyclase by NO) compared with poorly ventilated lung areas.

It can be stated on the basis of the findings which have been obtained that guanylate cyclase activators are able to enhance, in the sense of physiological adaptation of ventilation and perfusion, the necessary vasodilatation specifically in the well-ventilated regions in that they accentuate the physiological inhomogeneity of cGMP distribution in the lung and thus promotes rematching. Gas exchange is intensified and the oxygen supply is improved by this mechanism. Guanylate cyclase activators thus make selective relaxation of pulmonary vessels possible at the site of adequate ventilation.

A mismatch of pulmonary ventilation and pulmonary perfusion—up to the extremes of dead space ventilation and the shunting—may be caused by various inflammatory and degenerative lung disorders.

This mismatch may be present even at rest but may also appear only under conditions of increased ventilation and perfusion (meaning during exercise) (stress failure of the mismatch).

A patient according to this invention is a human. Patient preferably relates to a person requiring medical management or treatment.

The invention thus relates to the use of guanylate cyclase activitators for producing medicaments for the treatment of respiratory failure in patients with an exercise-dependent mismatch.

The phenomenon of exercise-induced ventilation/perfusion inhomogeneity occurs not only when there are underlying lung disorders, but also during normal aging processes (aging). However, in contrast to inflammatory and degenerative lung disorders, the main feature of age-related mismatch is an increasing rigidity of the pulmonary vessels, resulting in loss of the adaptation-optimizing physiological reflexes (hypoxic vasoconstriction). The mode of action of guanylate cyclase activators in these cases derives preferentially from the regionally selective vasodilating effect of the substances and the augmentation of the physiological residual signal (endogenous NO/prostacyclin).

The invention further relates to the use of guanylate cyclase activators for producing medicaments for the treatment of respiratory failure in patients with an age-related mismatch.

The invention further relates to the use of guanylate cyclase activators for producing medicaments for the treatment of respiratory failure in patients with a pathologically caused mismatch.

Patients with a pathologically caused mismatch are patients with a disorder selected from the group consisting of orthopnoea, sleep apnoea and COPD.

The use of guanylate cyclase activators is suitable specifically in patients with elevated low-V/Q perfusion (V/Q<0.1) to make physiological adaptation (rematching) of pulmonary ventilation and pulmonary perfusion possible through selective vasodilatation at the site of adequate ventilation. According to this invention, an elevated low-V/Q perfusion relates to areas of the lung in which ventilation is low but perfusion is good. A V/Q ratio can be determined in patients with an elevated low-V/Q perfusion through gas exchange measurements by means of MIGET.

The invention further relates to the use of guanylate cyclase activators for producing medicaments for the treatment of respiratory failure in patients with a V/Q of <0.1.

The invention further relates to the use of guanylate cyclase activators in the production of medicaments for the treatment of COPD patients. COPD patients with a V/Q of <0.1 are preferred.

Particular preference is given to treating COPD patients with a predominating bronchitic component (0.001<V/Q<0.1).

COPD patients with a predominanting bronchitic component (called “blue bloaters”) are distinguished by the presence of low-V/Q areas. Guanylate cyclase activators contribute to rematching in this subgroup of patients through the predominant vasodilatation in the remaining ventilated areas of the lung.

The invention further relates to the use of guanylate cyclase activators in the production of medicaments for the treatment of COPD patients with an emphysematous component. Preference is given to COPD patients with an emphysematous component with a V/Q of >10.

COPD patients with a predominating emphysematous component (called “pink puffers”) are distinguished by the presence of high-V/Q areas and increased dead-space ventilation as the cause of their mismatch. Guanylate cyclase activators can contribute to rematching in these patients because of an enhancement of perfusion in the hyperventilated areas (normalization of the V/Q ratio).

The invention additionally relates to the use of guanylate cyclase activators in the production of medicaments for the treatment of patients with orthopnoea. Preference is given to those patients suffering from posture-dependent impairments of gas exchange (orthopnoea) with nocturnal desaturation phases.

In a particular group of patients with manifest or latent respiratory failure there is a deterioration in gas exchange on passing from the vertical to the horizontal position (supine position). The change in position results in a redistribution of the ventilation distribution and also of the perfusion distribution, which are only poorly matched in these patients. The limited adaptation capacity means that the matching and correspondingly the O₂ saturation is reduced. This phenomenon is characterized clinically as orthopnoea. The patient develops critical phases of hypoxia, especially during periods of sleep, with the danger of unnoticed undersupply of oxygen, especially to the brain and myocardium. Guanylate cyclase activators are able, owing to their rematching effect, to increase the O₂ saturation in these patients and to reduce the risk of secondary organ damage.

The invention further relates to the use of guanylate cyclase activators in the production of medicaments for the treatment of patients suffering from sleep apnoea.

According to this invention, sleep apnoea is a nocturnal disturbance of respiratory regulation in which arterial hypoxia develops. These patients differ from other patients in that, owing to failure of the central respiratory drive or owing to anatomically caused peripheral obstruction (tongue closes the upper airways), alveolar ventilation is restricted and alveolar hypoxia is induced. The hypoxic vasoconstriction induced thereby with a subsequent rise in the pulmonary vascular resistance and severe stress on the right heart leads to damage to the myocardium (cor pulmonale) and to the blood vessels (essential hypertension). Administration of conventional vasodilators can certainly dilate the pulmonary vessels and thus reduce the stress on the right heart, but at the cost of a further deterioration in the already impaired gas exchange function through aggravation of the mismatch. Administration of guanylate cyclase activators thus makes it possible simultaneously to reduce the pulmonary vascular resistance and to prevent or reduce the mismatch.

The invention further relates to the use of guanylate cyclase activators in the production of medicaments for the treatment of a therapy-indiced mismatch.

In the treatment of patients with respiratory failure with β2 agonists, theophylline or systemic vasodilators (endothelin antagonists, Ca channel blockers, ACE inhibitors, ATII antagonists, β blockers) there is enhancement of a mismatch which is present. Although the vascular resistance in the lung is reduced on treatment with these medicines, simultaneously the O₂ saturation is reduced. This loss of O₂ saturation increasingly reduces the functional capacity of a patient which is already limited. Consequently, a latent or manifest respiratory failure may be induced in these patients through intake of nonselective vasodilators which is necessary to treat other disorders (therapy-induced mismatch). Guanylate cyclase activators are suitable for treating this type of respiratory failure.

Preference is given to uses of guanylate cyclase activators for the treatment of a therapy-induced mis-match on administration of nonselectively vasodilating medicaments, especially nonselectively vasodilating antiobstructive agents. According to this invention, the nonselectively vasodilating antiobstructive agent is selected from the group consisting of endothelin antagonist, Ca-channel blocker, ACE inhibitor, ATII antagonist and β blocker.

This invention further relates to the use of guanylate cyclase activators for producing medicaments for the treatment of muscular dysfunction caused by perfusion/demand mismatch.

In skeletal muscles (including the respiratory muscle) there is a stress-controlled adaptation of perfusion to the regional energy demand. Regulation of this “perfusion/demand matching” takes place in analogy to the lung through local release of endogenous vasodilators (especially NO/cGMP). The demand-oriented perfusion favours the stressed muscle groups (muscular selectivity), and within the muscle groups favours the specifically stressed fibre types (intramuscular selectivity). The type of stress, duration of stress and level of stress thus determine under physiological conditions the specific perfusion profiles in each case. Various inflammatory disorders (COPD, interstitial lung disorders, infections, vasculitides, degenerative vascular disorders, metabolic disorders), but also the use of nonselective vasoactive medicines for the treatment of the abovementioned disorders, may lead to a perfusion/demand mismatch. The consequence Is wasted perfusion of unstressed muscle groups to the detriment of perfusion of stressed muscle groups, with the result of a limitation on muscular performance. Guanylate cyclase activators are able to augment the physiological NO/cGMP distribution pattern and thus achieve muscular rematching.

This invention further relates to a pharmaceutical preparation comprising at least one guanylate cyclase activator and at least one nonselectively vasodilating antiobstructive agent. Such a combination is preferred for the treatment of partial and global respiratory failure. Such a combination is particularly preferred for the treatment of disorders selected from the group consisting of COPD, bronchial asthma, latent pulmonary hypertension associated with underlying lung disorder, emphysema, combined ventilation disturbances, chronic left heart failure with pulmonary congestion.

According to this invention, the nonselectively vasodilating antiobstructive agent is selected from the group consisting of endothelin antagonist, Ca channel blocker, ACE inhibitor, ATII antagonist and β blocker.

Examples of endothelin antagonists which may be mentioned are the compounds (2R,3R,4S)-1-[(dibutylcarbamoyl)methyl]-2-(p-methoxyphenyl)-4-(3,4-(methylenedioxy)phenyl]-3-pyrrolidinecarboxylic acid; N-(3,4-dimethyl-5-isoxazolyl)-4′-(2-oxazolyl)-[1,1′-biphenyl]-2-sulfonamide; p-tert-butyl-N-[6-(2-hydroxyethoxy)-5-(o-methoxyphenoxy)-2(2-pyrimidinyl)-4-pyrimidinyl]benzenesulfonamide; (+)-2(S)-(4,6-dimethylpyrimidin-2-yloxy)-3,3-diphenylbutyric acid; 2(S)-(4,6-dimethoxypyrimidin-2-yloxy)-2-yloxy)-3-methoxy-3,3-diphenyl-propionic acid; N-[2′-(4,5-dimethylisoxazol-3-ylsulfamoyl)-4-(2-oxazolyl)biphenyl-2-yl-methyl]-N,3,3-trimethylbutyramide; (5S,6R,7R)-2-butyl-7-[2-[2(S)-carboxypropyl]-4-methoxyphenyl]-5-(3,4-methylenedioxyphenyl)-6,7-dihydro-5H-cyclopenta[b]pyridine-6-carboxylic acid; (+)-(1S,2R,3S)-3-(2-carboxymethoxy-4-methoxyphenyl)-1-(3,4-methylenedioxyphenyl)-5-(prop-1-yloxy)indane-2-carboxylic acid; N-(4-chloro-3-methylisoxazol-5-yl)-2-[2-(6-methyl-1,3-benzodioxol-5-yl)-acetyl]thiophene-3-sulfonamide; N-(2-acetyl-4,6-dimethylphenyl)-3-[N-(3,4-dimethylisoxazol-5-yl)]-sulfamoyl]thiophene-2-carboxamide; N-[6-(2-hydroxyethoxy)-5-(o-methoxyphenoxy)-2-[2-(1H-tetrazol-5-yl)-4-pyridyl]-4-pyrimidinyl]-5-(1-isopropyl)-2-pyridinesulfonamide; and (E)-N-[6-methoxy-5-(2-methoxyphenoxy)-2-(pyrimidin-2-yl)pyrimidin-4-yl]-2-phenylethenesulfonamide; N-(3-methoxy-5-methylpyrazinyl)-2-[4-(1,3,4-oxadiazol-2-yl)phenyl]-3-pyridinesulfonamide.

Examples of Ca channel blockers which may be mentioned are the compounds 3-ethyl 5-methyl (plus/minus)-2-[(2-aminoethoxy)methyl]4-(o-chlorophenyl)-1,4-dihydro-6-methyl-3,5-pyridinedicarboxylate; (+)-5-[2-(dimethylamino)ethyl]-cis-2,3-dihydro-3-hydroxy-2-(4-methoxyphenyl)-1,5-benzothiazepin-4-(5H)-one acetate; 1-(diphenylmethyl)-4-[3-(2-phenyl-1,3-dioxolan-2-yl)propyl]piperazine; 1-[[p-[3-[(3,4-dimethoxyphenethyl)methylamino]propoxy]phenyl]sulfonyl]-2-isopropylindolizine; 1-(5-isoquinolinesulfonyl)hexahydro-1H-1,4-diazepine; (plus/minus)-ethyl methyl 4-(2,3-dichlorophenyl)-1,4-dihydro-2,6-dimethyl-3,5-pyridinedicarboxylate; (plus/minus)-3-(4-allyl-1-piperazinyl)-2,2-dimethyl methyl 1,4-dihydro-2,6-dimethyl-4-(m-nitrophenyl)-3,5-pyridinedicarboxylate; 4-[3,6-dihydro-6-hydroxy-5-[4-methyl-6-(2,6,6-trimethyl-1-cyclohexen-1-yl)-3-hexenyl]-2H-pyran-2-yl]-5-hydroxy-2(5H)-furanone; dimethyl 1,4-dihydro-2,6-dimethyl-4-(2-nitrophenyl)pyridine-3,5-dicarboxylate; 1,4-dihydro-2,6-dimethyl-4-(o-nitrophenyl)-3,5-pyridinedicarboxylic acid, dimethyl ester; (E)-cinnamyl-methyl (plus/minus)-1,4-dihydro-2,6-dimethyl-4-(m-nitrophenyl)-3,5-pyridinedicarboxylate; N-(2,6-dimethylphenyl)-4-[2-hydroxy-3-(2-methoxyphenoxy)propyl]-piperazine-1-acetamide; (plus/minus)-4-(2-Benzothiazolylmethylamino)-alpha[(4-fluorphenoxy)methyl]-1-piperidin ethanol; (+)-(R)-2-[5-methoxy-2-[3-[methyl[2-[3,4-(methylenedioxy)-phenoxy]-ethyl]amino]propoxy]phenyl]-4-methyl-2H-1,4-benzothiazin-3(4H)-one; and alpha-[3-[[2-(3,4-dimethoxyphenyl)ethyl]methylamino]propyl]-3,4-dimethoxy-alpha-(1-methylethyl)benzene-acetonitrile.

Examples of ACE inhibitors (angiotensin converting enzyme inhibitors) which may be mentioned are the compounds 1-carboxymethyl-3-[1-ethoxycarbonyl-3-phenyl-(1S)-propylamino]-2,3,4,5-tetrahydro-1H-1(3S)-benzazepin-2-one; 1-[(2S)-3-mercapto-2-methylpropionyl]-L-proline; (S)-1-[N-[1-(ethoxycarbonyl)-3-phenylpropyl]-L-alanyl]-L-proline; (4S)-4-cyclohexyl-1-[[(R-[(S)-1-hydroxy-2-methyl-propoxy]-(4-phenyl-butyl)phosphinyl]acetyl]-L-proline propionate (ester); (3S)-2-[(S)-N-[(S)-1-carboxy-3-phenylpropyl]alanyl]-1,2,3,4-tetrahydro-3-isoquinolinecarboxylic acid; and (4S)-N-[(S)-3-mercapto-2-methylpropionyl]-4-(phenylthio)-L-proline benzoate (ester).

Examples of ATII antagonists (angiotensin II antagonisten) which may be mentioned are the compounds 2-butyl-6-(1-methoxy-1-methylethyl)-3-[2′-(1H-tetrazol-5 yl)biphenyl-4-ylmethyl]quinazolin-4(3H)-one; 2-butyl-1-[2′-(1H-tetrazol-5-yl)biphenyl-4-ylmethyl]spiro[2-imidazoline-4,1′-cyclopentane]-5-one; 2-[[5-ethyl-3-[2′-(1H-tetrazol-5-yl)biphenyl-4-ylmethyl]-2,3-dihydro-1,3,4-thiadiazol-2ylidene]aminocarbonyl]-1-cyclopentenecarboxylic acid; methyl 2-[[4-butyl-2-methyl-6-oxo-5-[p-(o-1H-tetrazol-5-ylphenyl)benzyl]-1(6H)-pyrimidinyl]methyl]-3-thiophenecarboxylate; 4-(1-hydroxy-1-methylethyl)-2-propyl-1-[2′-(1H-5-tetrazolyl)biphenyl-4-yl-methyl]imidazole-5-carboxylic acid 5-methyl-2-oxo-1,3-dioxol-4-yl-methyl ester; 1-[3-bromo-2-[2-(trifluoromethylsulfonamido)phenyl]-benzofuran-5-yl-methyl]-4-cyclopropyl-2-ethyl-1H-imidazole-5-carboxamide; 2-Ethoxy-1-[2′-(5-oxo-2,5-dihydro-1,2,4-oxadiazol-3-yl)biphenyl-4-yl-methyl]benzimidazole-7-carboxylic acid; 2,4-dimethyl-8-[2′-(1H-tetrazol-5-yl)biphenyl-4-ylmethyl]-5,6,7,8-tetrahydropyrido[2,3-d]pyrimidin-7-one; 4′-[(2-n-propyl-4-methyl-6-(1-methylbenzimidazol-2-yl)-benzimidazol-1-yl)methyl]biphenyl-2-carboxylic acid; N-pentanoyl-N-[2′-(1H-tetrazol-5-yl)-biphenyl-4-ylmethyl]-L-valine; and 1-[3-bromo-2-[2-(1H-tetrazol-5-yl)phenyl]benzo[b]furan-5 ylmethyl]-2-butyl-4-chloroimidazole-5-carboxylic acid.

Examples of β blockers which may be mentioned are the compounds 2-[p-[2-hydroxy-3-isopropylamino)propoxy]phenyl]acetamide; (plus/minus)-1-[[2-(3,4-dimethoxyphenyl)ethyl]amino]-3-(3-methylphenoxy)-2-propanol; (plus/minus)1-(tert-butylamino)-3-[(2-methylindol-4-yl)oxy]-2-propanol benzoate (ester); 1-(9H-carbazol-4-yloxy)-3-[(1-methylethyl)amino]-2-propanol; (plus/minus)-1-(carbazol-4-yloxy)-3-{[2-(o-methoxyphenoxy)ethyl]amino)-2-propanol; (−)-5-[3-(tert-butylamino)-2-hydroxypropoxy]-3,4dihydro-1(2H)-naphthalenone; 1-(isopropylamino)-3-[(2-methylindol-4-yl)oxy]-2-propanol; (plus/minus)-1-[4-(2-methoxyethyl)phenoxy]-3-[(1-methylethyl)amino]-2-propanol; 1-[o-(Allyloxy)phenoxy]-3-(isopropylamino)-2-propanol; 4-[2-hydroxy-3-(isopropylamino)propoxy]indole; N-[4-[1-hydroxy-2-[(1-methylethyl)amino]ethyl]phenyl]-methanesulfonamide; and (−)-1-(tert-butylamino)-3-[(4-morpholino-1,2,5-thiadiazol-3-yl)oxy]-2-propanol.

Nonselectively vasodilating antiobstructive agents are used in medicaments for the treatment of obstructive ventilation impairment. Administration of such antiobstructive agents may considerably exacerbate the disturbance of gas exchange function, caused by a nonselective vasodilatation—especially in the poorly ventilated lung areas—which may lead to an increase in mismatch and shunting. Guanylate cyclase activators are able to show their selective effect also in combination with nonselectively vasodilating antiobstructive agents and, through their selective effect, compensate the mismatch caused by the nonselectively vasodilating antiobstructive agents. Nonselectively vasodilating antiobstructive agents and Guanylate cyclase activators can be administered in a fixed combination. It is likewise possible to administer nonselectively vasodilating antiobstructive agents and Guanylate cyclase activators as free combination—singly—in which case administration can take place in immediate succession or at a relatively large time interval. According to this invention, a relatively large time interval relates to a time interval of up to a maximum of 24 hours.

Substances which can be included among guanylate cyclase activators for example are those described and claimed in the following patent applications and patents: WO0183490, WO0117998, WO0006569, EP0515420, DE2145359, DE2410201, EP0359335, WO0027394, WO9401422, EP0210581, EP0490183, DE3134929, EP0388528, EP0490183, EP0326575, EP0451760, EP0044927, EP0667345, DE3706731, EP0147193 and WO8912069.

Examples of guanylate cyclase activators which may be mentioned are the compounds (9S,11S)-4-amino-2-[1-(2-fluorobenzyl)-1H-pyrazolo[3,4-b]pyridin-3-yl]-6,7,9,10-tetrahydro-5,9-methan-opyrimido[4,5-d][1,3,6]oxadiazocin-11-ol, 5-cyclopropyl-2-[1-(2-fluorobenyl)-1H-pyrazolo[3,4-b]pyridin-3-yl]-pyrimidine-4-amine (BAY-41-2272), 2-[1-(2-fluorobenzyl)-1H-pyrazolo[3,4-b]pyridin-3-yl]-5-(4-morpholinyl)pyrimidine-4,6-diamine (BAY-41-8543), trans-4-acetamidocyclohexyl nitrate (BM-12.1307), 1-[2-(1,3-dimethyl-2-butenylidene)hydrazino]-phthalazine (BUDRALAZINE), ethyl 3-{6-[N-(2-hydroxypropyl)-N-ethylamino]-3-pyridazinyl}-carbazate (CADRALAZINE), trans-1,4-di(hydroxymethyl)cyclohexane dinitrate ester (CEDO-8956), 1-benzyl-3-[3-(dimethylamino)propoxy]-N-(4-methoxyphenyl)-1H-pyrazole-5-carboxamide (CFM-1571), 4-phenyl-3-(phenylsulfonyl)furazan 2-oxide (CHF-2206), 8-hydroxy-2-(nitratomethyl)-7-nitro-1,4-benzodioxane (E-4701), 1,2,3-propanetriyl trinitrate (GLYCEROL TRINITRATE), 1,4:3,6-dianhydro-D-glucitol dinitrate (ISOSORBIDE DINITRATE), 1,4:3,6-dianhydro-D-glucitol 5-mononitrate (ISOSORBIDE MONONITRATE), 6-methyl-3-[2-(nitrooxy)-ethyl]-3,4-dihydro-2H-1,3-benzoxazin-4-one (ITF-1129), 7-[2-[4-(2-chlorophenyl)piperazin-1-yl]ethyl]-1,3-dimethyl-3,7-dihydro-1H-purine-2,6-dione (KMUP-1), N-cyano-N′-(2-nitrooxyethyl)-3-pyridinecarboxamidine (KRN-2391), 3-(2-nitrooxyethyl)-3,4-dihydro-2H-1,3-benzoxazin-4-one (SINI-TRODIL), sodium pentacyanonitrosylferrate(II) (SODIUM NITROPRUSSIDE), N-(3-nitratopivaloyl)cysteine ethyl ester (SPM-3672), 3-[2-(acetamido)propionylthio]-2(R)-[2,2-dimethyl-3-(nitrooxy)propionamido]propionic acid ethyl ester (SPM-5185), 1,4:3,6-dianhydro-2-deoxy-2-[[3-(1,2,3,6-tetrahydro-1,3-dimethyl-2,6-dioxopurin-7-yl)propyl]amino]-L-iditol 5-nitrate (TEOPRANITOL), glutaminyl-glutamyl-aspartyl-cysteinyl-glutamyl-leucyl-cysteinyl-isoleucyl-asparaginyl-valyl-alanyl-cysteinyl-threonyl-glycyl-cysteine (UROGUANYLINE), 1-benzyl-3-[5-(hydroxymethyl)furan-2-yl]-1H-indazole (YC-1), L-arginyl-L-seryl-L-seryl-L-cysteinyl-L-phenyalanyiglycylglycyl-L-arginyl-L-methionyl-L-aspartyl-L-arginyl-L-isoleucyl-glycyl-L-alanyl-L -glutaminyl-L-serylglycyl-L-leucylglycyl-L-cysteiny-L-asparaginyl-L-seryl-L-phenylalanyl-L-arginyl-L-tyrosine, cyclic (4→20) disulfide (ANARITIDE), 1-de-L-leucine-2-de-L-alanine-3-deglycine-4-de-L-proline-5-de-L-arginine-17-L-methionine-atriopeptin-33 (rat) (CARPERITIDE), seryl-prolyl-lysyl-methionyl-valyl-glutaminyl-glycyl-seryl-glycyl-cysteinyl-phenylalanyl-glycyl-arginyl-lysyl-methionyl- aspartyl-arginyl-isoleucyl-seryl-seryl-seryl-seryl-glycyl-leucyl-glycyl-cysteinyl-lysyl-valyl-leucyl-arginyl-arginyl-histidine, cyclic (S-3.10-S-3.26)-disulfide (NESIRITIDE), 4-[N-(4-carboxybutyl)-N-[2-[2-[4-(2-phenylethyl)benzyloxy]phenyl]-ethyl]aminomethyl]benzoic acid, 3-[2-(4-chlorophenylsulfanyl)-phenyl]-N-[4-(dimethylamino)butyl]-2-propanamide, 3-[2-(4-chlorophenylsulfanyl)phenyl]-N-[4-(dimethylamino)butyl]-2-propenamide, 3-(5′-Hydroxymethyl-2′-furyl)-1-benzyl indazole (YC-1) and the pharmacologically acceptable salts of these compounds.

Particularly preferred guanylate cyclase activators are those selected from the group consisting of BM-12.1307, BUDRALAZINE, CADRALAZINE, GLYCEROL TRINITRATE, ISOSORBIDE DINITRATE, ISOSORBIDE MONONITRATE, KRN-2391, SINITRODIL, SODIUM NITROPRUSSIDE, TEOPRANITOL, ANARITIDE, CARPERITIDE, NESIRITIDE new and the pharmacologically acceptable salts of these compounds.

Suitable salts are—depending on the substitution and depending on the basic structure—in particular all acid addition salts or else salts with bases. Particular mention may be made of the pharmacologically acceptable salts of the inorganic and organic acids normally used in pharmaceutical technology. Suitable as such are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in the preparation of salts—depending on whether the acid is monobasic or polybasic and depending on which salt is desired—in the equimolar ratio of amounts or one differing therefrom. Particular mention should also be made of the pharmacologically acceptable salts of the inorganic and organic bases normally used in pharmaceutical technology. Suitable as such are water-soluble and water-insoluble salts with bases such as, for example, sodium hydroxide solution, potassium hydroxide solution or ammonia.

In the use according to the Invention of guanylate cyclase activators for producing the aforementioned medicaments and in the pharmaceutical preparations according to the invention, the guanylate cyclase activators (=the active ingredients) are processed with suitable pharmaceutical excipients or carriers to tablets, coated tablets, capsules, suppositories, plasters (e.g. as transdermal therapeutic system=TTS), emulsions, suspensions or solutions, with the active ingredient content advantageously being between 0.1 and 95%, and it being possible by appropriate choice of the excipients and carriers to obtain a pharmaceutical dosage form (e.g. a slow-release form or an enteric form) which is exactly adapted to the active ingredient and/or to the desired onset of action.

Excipients and carriers suitable for the desired pharmaceutical formulations are familiar to the skilled person on the basis of his expert knowledge. Besides solvents, gel formers, suppository bases, tablet excipients and other active ingredient carriers it is possible to use, for example, antioxidants, dispersants, emulsifiers, antifoams, masking flavours, preservatives, solubilizers, colours or, in particular, permeation promoters and complexing agents (e.g. cyclodextrins).

The active ingredient can be administered orally, by inhalation, percutaneously or intravenously.

It has generally proved advantageous in human medicine to administer the active ingredient in the case of oral administration in a daily dose of about 0.02 to about 4 mg, in particular 0.1 to 2 mg per kg of body weight, where appropriate in the form of a plurality of, preferably 1 to 3, individual doses to achieve the desired result, with gradually increasing and decreasing dosage possibly being advantageous. On parenteral treatment it is possible to use similar or (especially on intravenous administration of the active ingredient) usually lower dosages.

The skilled person is aware that the optimal dose of an active ingredient may vary depending on the body weight, the age and the general condition of the patient, and on his response to the active ingredient.

Every skilled person is easily able to establish on the basis of his expert knowledge the optimal dosage and mode of administration of the active ingredient necessary in each case.

The invention further relates to a method of treating partial and global respiratory failure in a human in need thereof comprising the step of administering to said human a therapeutically effective amount of a guanylate cyclase activator.

According to this invention, a therapeutically effective amount of a guanylate cyclase activator refers to the pharmacologically tolerable amount of the guanylate cyclase activator sufficient, either as a single dose or as a result of multiple doses, to decrease the mismatch of pulmonary ventilation and pulmonary perfusion, or to reduce wasted perfusion and wasted ventilation.

The invention further relates to a method of treating respiratory failure in a human showing a mismatch of pulmonary ventilation and pulmonary perfusion comprising the steps of administration to said human in need a therapeutically effective amount of a guanylate cyclase activator. In particular, a method of treating respiratory failure in such a human with a mismatch of V/Q<0.1 is preferred.

The invention further relates to a method of treating respiratory failure in a human showing an exercise-dependent mismatch of pulmonary ventilation and pulmonary perfusion comprising the steps of administration to said human in need a therapeutically effective amount of a guanylate cyclase activator.

The invention further relates to a method of treating respiratory failure in a human showing an age-related mismatch of pulmonary ventilation and pulmonary perfusion comprising the steps of administration to said human in need a therapeutically effective amount of a guanylate cyclase activator.

The invention further relates to a method of treating respiratory failure in a human showing pathologically caused mismatch of pulmonary ventilation and pulmonary perfusion comprising the steps of administration to said human in need a therapeutically effective amount of a guanylate cyclase activator.

The invention further relates to a method of treating respiratory failure in a COPD patient comprising the steps of administration to said COPD patient a therapeutically effective amount of a guanylate cyclase activator.

The invention further relates to a method of treating respiratory failure in a COPD patient with a predominant bronchitis component comprising the steps of administration to said COPD patient a therapeutically effective amount of a guanylate cyclase activator.

The invention further relates to a method of treating respiratory failure in a COPD patient with a mismatch of V/Q<0.1 comprising the steps of administration to said COPD patient a therapeutically effective amount of a guanylate cyclase activator.

The invention further relates to a method of treating respiratory failure in a COPD patient with an emphysematous component comprising the steps of administration to said human in need a therapeutically effective amount of a guanylate cyclase activator.

The invention further relates to a method of treating respiratory failure in a COPD patient with a mismatch of V/Q>10 comprising the steps of administration to said COPD patient a therapeutically effective amount of a guanylate cyclase activator.

The invention further relates to a method of treating orthopnoea in a human showing a mismatch of pulmonary ventilation and pulmonary perfusion comprising the step of administering to said human a therapeutically effective amount of a guanylate cyclase activator.

The invention further relates to a method of treating sleep apnoea in a human showing a mismatch of pulmonary ventilation and pulmonary perfusion comprising the step of administering to said human a therapeutically effective amount of a guanylate cyclase activator.

The invention further relates to a method of treating respiratory failure in a human showing a therapy-induced mismatch comprising the steps of administering to said human in need a therapeutically effective amount of a selective guanylate cyclase activator.

The invention further relates to a method of treating respiratory failure in a human showing a mismatch caused by administration of nonselectively vasodilating medicaments, the method comprises the steps of administering to said human in need a therapeutically effective amount of a guanylate cyclase activator. In particular, such a method is preferred, wherein the nonselectively vasodilating medicament is a nonselectively vasodilating antiobstructive agent. The method is particularly preferred, wherein the non-selectively vasodilating antiobstructive agent is selected from the group consisting of endothelin antagonist, Ca channel blocker, ACE inhibitor, ATII antagonist and β blocker.

The invention further relates to a method of treating muscular dysfunction in a human showing a perfusion/demand mismatch comprising the step of administering to said human a therapeutically effective amount of a guanylate cyclase activator.

Further advantages and embodiments of the invention are described below and are evident from the examples and the appended drawings.

### DESCRIPTION OF THE FIGURES

FIG. 1:

Effect of the guanylate cyclase activator YC-1 (3-(5′-Hydroxymethyl-2′-furyl)-1-benzyl indazole) on NO-induced pulmonary vasodilatation. 10, 50, 100 and 200 ppm NO were administered cumulatively by inhalation in the model of U46619 (thromboxane agonist)-induced pulmonary hypertension in the isolated rabbit lung. This led to a dose-dependent reduction in the pulmonary arterial pressure. In another test group, after adjustment of the pulmonary hypertension by U46619, initially 0.1 μM YC-1 was administered. This dosage had no intrinsic effect on the pulmonary arterial pressure. Subsequently, 10, 50, 100 and 200 ppm NO were administered by inhalation. A significantly greater reduction in the pulmonary arterial pressure was achieved with the dosages of 50, 100 and 200 ppm NO. The average of 6 tests with standard error is depicted.

p<0.05 NO/YC-1 versus NO

FIG. 2:

Effect of inhaled NO on the pulmonary arterial pressure in the whole animal model (rabbit) of oleic acid-induced lung damage. Injection of 50 μg of oleic acid (OA) in the whole animal model of the anaesthetized and ventilated rabbit induced acute pulmonary hypertension which was accompanied by severe gas exchange impairments. Administration of 10, 34, 70, 120 and 200 ppm NO by inhalation led to a dose-dependent reduction in the pulmonary arterial pressure. In another test group, the oleic acid injection was followed by infusion of 5 mg of YC-1 over a period of 10 min. This dose had no effect on the measured parameters. Subsequently, 10, 34, 70, 120 and 200 ppm NO were administered by inhalation. The effect of NO was enhanced without a significant effect on the systemic arterial pressure. Average of n=6 tests with standard error.

FIG. 3:

Effect of inhaled NO on the systemic arterial pressure in the whole animal model (rabbit) of oleic acid-induced lung damage. Injection of 50 μg of oleic acid (OA) in the whole animal model of the anaesthetized and ventilated rabbit induced acute pulmonary hypertension which was accompanied by severe gas exchange impairments. Administration of 10, 34, 70, 120 and 200 ppm NO by inhalation did not lead to a dose-dependent reduction in the systemic arterial pressure. In another test group, the oleic acid injection was followed by infusion of 5 mg of YC-1 over a period of 10 min. This dose had no effect on the measured parameters. Subsequently, 10, 34, 70, 120 and 200 ppm NO were administered by inhalation. The effect of NO was enhanced without a significant effect on the systemic arterial pressure. Average of n=6 tests with standard error.

FIG. 4:

Effect of inhaled NO (OA/NO), of the guanylate cyclase activator YC-1 (OA/YC-1), and of a combination of NO and YC-1 (OA/NO/YC-1) on the pulmonary arterial pressure in the whole animal model (rabbit) of oleic acid-induced lung damage (OA). Administration of 100 ppm NO by inhalation from time 30 to 150 min led to a reduction in the pulmonary arterial pressure (OA/NO). In another test group, injection of oleic acid was followed by infusion of 5 mg of YC-1 for a period of 10 min (from time 30 to 40 min). This dose had no effect on the pulmonary arterial pressure (OA/YC-1). In a further test group, the infusion of YC-1 was combined with inhalation of 100 ppm NO, which led to an enhancement of the NO reduction of pulmonary arterial pressure. Average from n=6 tests with standard error.

*, p<0.05 versus the oleic acid-treated group (OA).

FIG. 5:

Effect of inhaled NO (OA/NO), of the guanylate cyclase activator YC-1 (OA/YC-1), and of a combination of NO and YC-1 (OA/NO/YC-1) on the intrapulmonary shunting in the whole animal model (rabbit) of oleic acid-induced lung damage (OA). Administration of 100 ppm NO by inhalation from time 30 to 150 min led to a reduction in the intrapulmonary shunting (measured by MIGET; group OA/NO). In another test group, injection of oleic acid was followed by Infusion of 5 mg of YC-1 for a period of 10 min (from time 30 to 40 min). This dose had no significant effect on intrapulmonary shunting (OA/YC-1). In a further test group, the infusion of YC-1 was combined with inhalation of 100 ppm NO, which led to a significant reduction in shunting. It was possible likewise to reduce significantly the shunting compared with the NO-treated group. Average from n=6 tests with standard error.

*, p<0.05 versus the oleic acid-treated group (OA).

$, p<0.05 versus the oleic acid/NO-treated group (OA/NO).

FIG. 6:

Effect of inhaled NO (OA/NO), of the guanylate cyclase activator YC-1 (OA/YC-1), and of a combination of NO and YC-1 (OA/NO/YC-1) on the arterial oxygenation in the whole animal model (rabbit) of oleic acid-induced lung damage 150 min after oleic acid injection (OA). Administration of 100 ppm NO by inhalation from time 30 to 150 min led to a significant increase in the arterial oxygenation (shown as ratio of arterial oxygenation to inspiratory oxygen concentration 150 min after oleic acid injection; OA/NO). In another test group, injection of oleic acid was followed by infusion of 5 mg of YC-1 for a period of 10 min (from time 30 to 40 min). This dose likewise had a significant effect on the oxygenation index (OA/YC-1). In a further test group, the infusion of YC-1 was combined with inhalation of 100 ppm NO, which led to a significant improvement in oxygenation. Average from n=6 tests with standard error.

*, p<0.05 versus the oleic acid-treated group (OA).

$, p<0.05 versus the oleic acid/NO-treated group (OA/NO).

FIG. 7:

Effect of inhaled NO (OA/NO), of the guanylate cyclase activator YC-1 (OA/YC-1) and of a combination of NO and YC-1 (OA/NO/YC-1) on the perfusion of normally ventilated areas of the lung (normal V/Q) in the whole animal model (rabbit) of oleic acid-induced lung damage (OA). Administration of 100 ppm NO by inhalation from time 30 to 150 min led to a significant increase in the perfusion of normally ventilated areas of the lung (normal V/Q [% Q]; measured by MIGET; group OA/NO). In another test group, injection of oleic acid was followed by infusion of 5 mg of YC-1 over a period of 10 min (from time 30 to 40 min). This dose had no significant effect on the perfusion of normally ventilated areas of the lung (OA/YC-1). In a further test group, infusion of YC-1 was combined with inhalation of 100 ppm NO, which led to a significant improvement in the perfusion of normally ventilated areas of the lung. This increase was likewise significant compared with the NO-treated animals. Average of n=6 tests with standard error.

*, p<0.05 versus the oleic acid-treated group (OA).

$, p<0.05 versus the oleic acid/NO-treated group (OA/NO).

### EXAMPLES

Tests were carried out on two experimental models. On the model of the isolated bloodlessly perfused and ventilated rabbit lung with U46619-induced acute pulmonary hypertension and on a whole animal model (rabbit) of acute lung damage with pulmonary hypertension due to injection of oleic acid (OA). The essential results are a) an enhancement of the reduction in pressure of inhaled nitric oxide (NO) in the presence of the guanylate cyclase activator YC-1 in the model of the isolated rabbit lung and b) enhancement of the pressure-lowering effect of NO by YC-1 and improvement of gas exchange with retention of the pulmonary selectivity in the model of oleic acid-induced acute lung damage in rabbits.

### Example 1

Model of the Isolated, Bloodlessly Perfused and Ventilated Rabbit Lung

The test animal was anaesthetized by injection of about 700 μl of a mixture of Ketanest and Rompun in the ratio 3:2. The spontaneous breathing of the animal was maintained with this initial anaesthesia. 1 000 I.U. of heparin per kg of bodyweight were injected through the venous access for anticoagulation. For the intubation, 7 ml of Xylocaine were injected into the subcutaneous tissue of the animal's neck. A tube was introduced into the trachea underneath the larynx and was used from this instant onwards to ventilate the animal with ambient air through the ventilating pump (breathing rate: 30 s⁻¹, tidal volume 30 ml). About 3 ml of the anaesthetic mixture were administered over a period of 15 min. The lungs were then removed by a standard technique; likewise dissection of the pulmonary artery and the ascending aorta.

To make it possible to remove the lung from the body's own perfusion without interruption, the pulmonary artery catheter of the perfusion system was, after incision of the right ventricle, advanced into the pulmonary artery and fixed there by means of the open ligature. After cutting off the apex of the heart and closing the ascending aorta, the lung was artificially perfused with Krebs-Henseleit buffer cooled to 4° C. (the cooling served to reduce metabolism) at 20 ml/min. The ambient air was replaced by a 5% CO₂, 15% O₂ and 80% N₂ gas mixture.

The lung was dissected out of the thorax, and a connector was sutured into the left heart to allow the perfusion circulation to be completed. The lung was suspended on a weighing cell and the perfusion circulation was completed through the connector. The lung perfusate then flowed out through a pressure cascade. The temperature of the complete system was raised to 38° C. and the pressure recording started. In the system, the pulmonary artery pressure, the left ventricular pressure, the ventilation pressure and the weight were recorded continuously. After connecting the perfusion circulation, the perfusion flow was increased over the course of 10 min to 120 m/min, and the left ventricular pressure was adjusted to 2 mmHg through the hydrostatic pressure level. At a flow rate of 120 ml/min, a perfusate change was carried out and the optional filter was bypassed. In addition, the expired air was passed through a positive end-expiratory pressure: (PEEP) of 1 cm H₂O. The lungs used for the tests were homogeneously white on the outside due to the perfusion and had no atelectases and no vessel leaks.

A sterile Krebs-Henseleit solution from Serag-Wiesner (Naila, FRG) with the following concentrations was used as perfusate: sodium chloride [145.0 mM], potassium dihydrogen phosphate [1.1 mM], magnesium chloride hexahydrate [1.3 mM], potassium chloride [4.3 mM], calcium chloride dihydrate [2.4 mM], glucose [13.3 mM] and hydroxyethyl starch [MW 200 000] [50 g/l].

It was possible to induce stable pulmonary hypertension by continuous intravascular administration of an amount of from 70 to 160 pmol/kg min U46619, a stable thromboxane analogue. This raised the pulmonary arterial pressure from 6-8 mmHg to a level of 33-35 mmHg. 15 min after starting the U46619 infusion, no further change in the infusion rate was necessary, and the Perfusor setting which had been adjusted was retained up to the end of the test.

Adjustment to the stable pressure level was followed by (1) administration of nitric oxide (NO) (admixed to the inspired air) in concentrations of 10, 50, 100 and 200 ppm (parts per million) and (2) intravenous administration of the guanylate cyclase activator YC-1 (3-(5′-Hydroxymethyl-2′-furyl)-1-benzyl indazole) in a concentration of 0.1 μM, followed by (3) inhalation of 10, 50, 100 and 200 ppm NO.

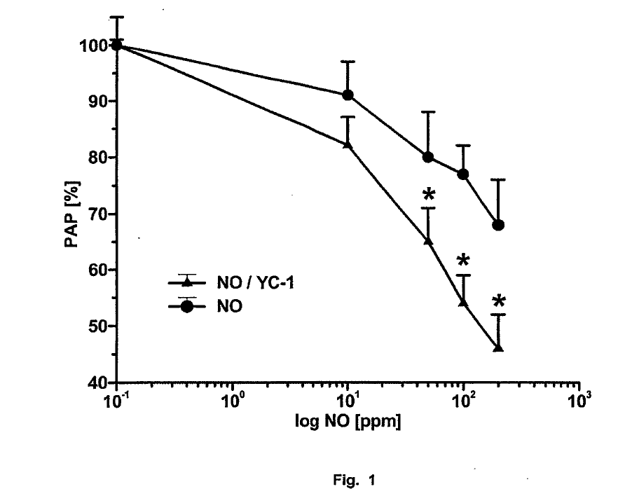

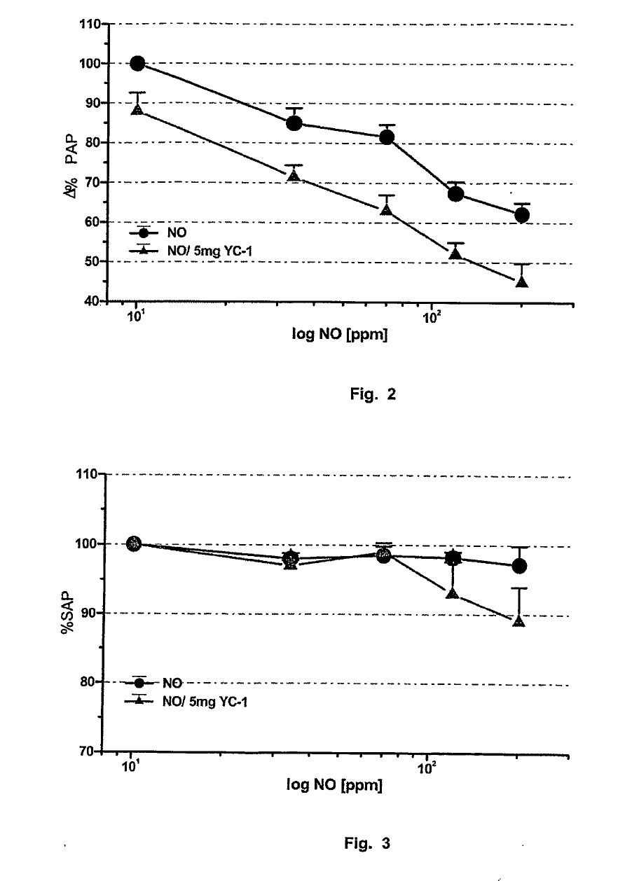

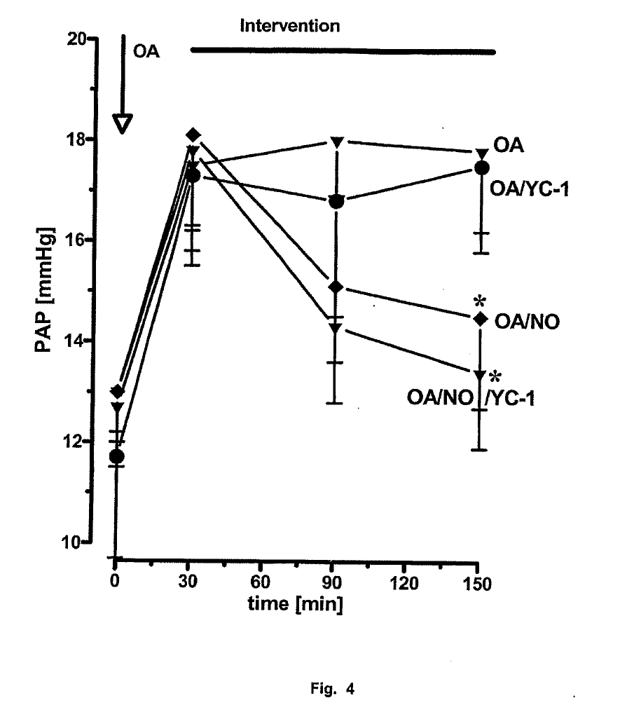

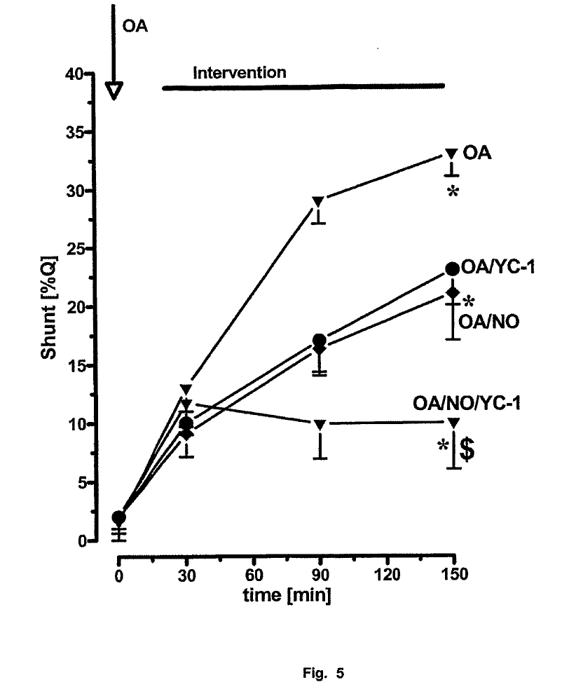

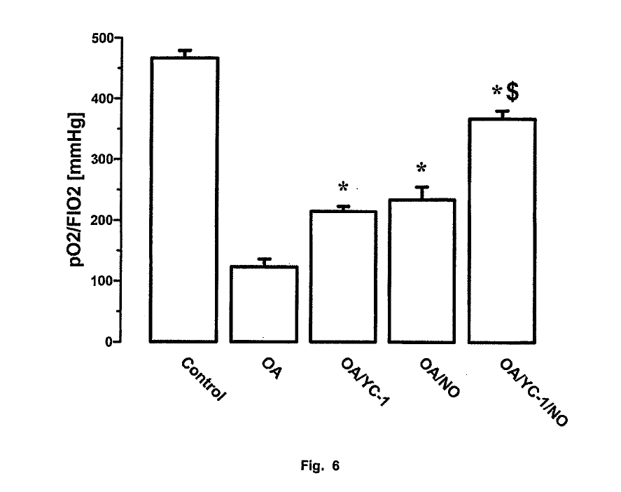

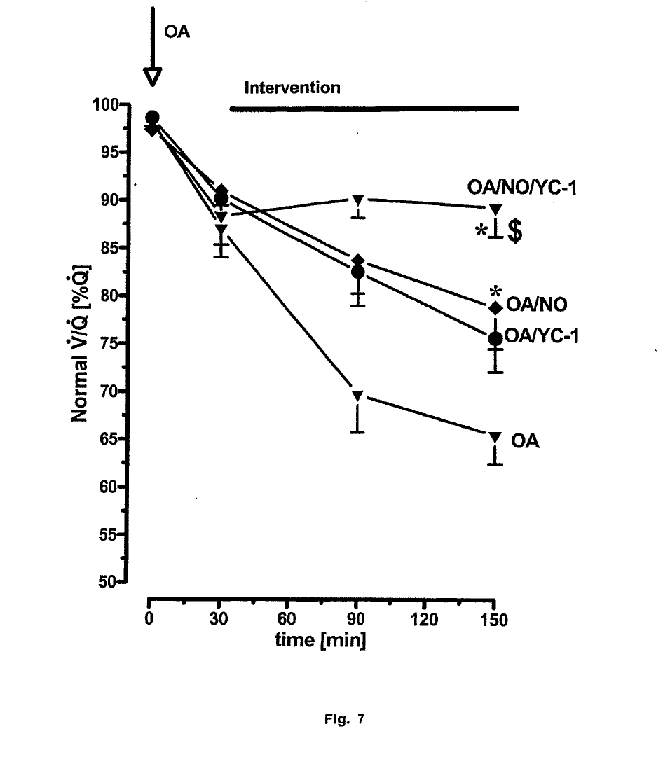

In the presence of the guanylate cyclase activator YC-1, the pulmonary arterial pressure reduction with inhaled NO in concentrations of 50, 100 and 200 ppm was significantly enhanced (FIG. 1).

### Example 2

Whole Animal Model of Acute Pulmonary Hypertension with Severe Gas Exchange Impairment Through Infusion of Oleic Acid

The test animals In the rabbit model were anaesthetized by injection of about 700 μl of a mixture of Ketanest and Rompun in the ratio 3:2. The spontaneous breathing of the animal was maintained with this initial anaesthesia. 1 000 I.U. of heparin per kg of bodyweight were injected for anticoagulation. For the intubation, 7 ml of Xylocaine were injected into the subcutaneous tissue of the animal's neck. A tube was introduced into the trachea underneath the larynx and was used from this instant onwards for ventilation of the animal through the ventilation pump (breathing rate: 25 s⁻¹, tidal volume 35 ml). The animal underwent standard ventilation with a 50% N₂ and a 50% O₂ gas mixture. 7 ml/h of the anaesthetic mixture were administered through a Perfusor, which led to a deep analgesia and relaxation of the animal. The left common carotid artery was ligated and punctured for measurement of the arterial pressure. In a next step, the superior vena cava was ligated and a port was introduced. Through this port, a 4F balloon catheter was placed in the right ventricle and the right ventricular pressure was recorded. The gas exchange was analysed by the multiple inert gas elimination technique (MIGET). The MIGET is based on elimination and retention of a plurality of inert gases. Central venous infusion thereof in dissolved form into the animal via the ear margin vein was therefore necessary. This infusion solution was prepared by introducing 250 ml of the perfusate without air bubbles into a gas-tight bag. 0.5 ml of liquid halothane was put into this bag through an injection plug. The bag was then filled with a test gas mixture (10% SF₆ 3.0, 20% cyclopropane 2.0 and 70% ethane 2.5) and the gases were dissolved in the perfusate. 0.15 ml of diethyl ether was injected, followed by 0.7 ml of acetone. This solution was infused continuously at 30 ml/h into the animal during the equilibration period after change of the perfusate. An equilibrium between retention and elimination of the gases was set up within a period of 30-40 min. After this time, 2.5 ml samples were taken simultaneously from the arterial and venous blood by gas-tight 50 ml glass syringes (B-D Yale, Becton, Dickinson & Co, USA). The arterial and the venous sample was then weighed, blanketed with 15 ml of nitrogen gas (ECD grade) and incubated at 38° C. in a shaking water bath (135 min⁻¹). During this time, an equilibrium, depending on the solubility of the gas, was set up between the gaseous and liquid phase. After the equilibration time had elapsed, the total volume of the syringe was determined and the supernatant gas was transferred into an air-tight 30 ml glass syringe (B-D Yale, Becton, Dickinson & Co, USA) preheated to 38° C. The latter was stored at 38° C. and used for gas chromatographic analysis. Immediately after removal of the arterial and venous perfusate, a sample of the expired air from the isolated lung was taken by a preheated 30 ml glass syringe on an expiratory gas mixing box. This gaseous sample was analysed immediately in a gas chromatograph. The gases were analysed by a computer-assisted calculation through which essentially the following parameters were calculated: shunting (perfusion, unventilated regions of the lung), normal V/Q (perfusion in normally ventilated regions of the lung).

Besides the MIGET data, the following haemodynamic parameters were determined:
pulmonary arterial pressure
systemic arterial pressure
arterial oxygen partial pressure (pO₂)

Catheterization of the animal was followed by injection of 50 μg of oleic acid (OA) into the pulmonary artery. The pulmonary arterial pressure rose after this injection from 11-13 mmHg to a stable plateau of 17-18 mmHg. This rise in the pulmonary arterial pressure was accompanied by severe gas exchange impairment essentially characterized by a fall in the arterial oxygenation and a rise in the shunt perfusion (measured by the MIGET). In this model, administration by inhalation of increasing dosages of nitric oxide (NO, 10, 34, 70, 120 and 200 ppm) then took place. The results are depicted in FIG. 2. A dose-dependent reduction in the pulmonary arterial pressure was possible in this case. The systemic arterial pressure remained unchanged (FIG. 3). In the presence of 5 mg of YC-1 (3-(5′-Hydroxymethyl-2′-furyl)-1-benzyl indazole, administered as brief infusion over 10 min) it was possible to enhance the effect of inhaled NO without a significant effect on the systemic arterial pressure.

Combination administrations over a period of 120 min took place in the same model. For this purpose, a dose of the guanylate cyclase activator YC-1 which per se had no effect on the pulmonary arterial pressure was infused over a period of 10 min and then combined with 100 ppm inhaled NO. The following test groups were carried out:
OA: oleic acid administration (50 μg i.v.) at time 0 min, injection and inhalation of placebo
OA/YC-1: oleic acid administration at time 0 min and subsequent brief infusion of 5 mg of YC-1 over 10 min from time 30 to 40 min.
OA/NO: oleic acid administration at time 0 min and subsequent inhalation of 100 ppm NO over 120 min from time 30 to 150 min.
OA/NO/YC-1: oleic acid administration at time 0 min followed by brief infusion of 5 mg of YC-1 over 10 min from time 30 to 40 min and inhalation of 100 ppm NO over 120 min from time 30 to 150 min.

Injection of oleic acid led to an increase in the pulmonary arterial pressure from 11-13 mmHg to a stable plateau of 17-18 mmHg (FIG. 4) and an increase in the intrapulmonary shunting to 30-35% (FIG. 5). These disturbances were accompanied by a fall in the arterial oxygenation (FIG. 6) and the perfusion of normally ventilated areas of the lung (normal V/Q) (FIG. 7). Administration of 5 mg of YC-1 led to no effect on the pulmonary arterial pressure, but it was possible to reduce the shunting from 33±2 to 21±4% (n.s.). The arterial oxygenation rose significantly from 123±13 to 215±8 mmHg. Inhalation of 100 ppm NO led to a significant reduction in the pulmonary arterial pressure from 17.8±1.6 to 14.5±1.8 mmHg and to an improvement in the gas exchange through a reduction in shunting (23±3 versus 33±2%) and improvement in oxygenation (234±21 versus 123±13 mmHg). The combination of inhalation of 100 ppm NO with intravenous administration of YC-1 led to a further reduction in the pulmonary arterial pressure to 13.4±1.5 mmHg and to a reduction, which was significant compared with the NO-treated group, in the shunting (10±4 versus 23±3%) and improvement in oxygenation (367±13 versus 234±21 mmHg). The perfusion of normally ventilated areas of the lung was likewise significantly increased versus the animals treated with NO alone.

1.-19. (canceled)

20. A pharmaceutical preparation comprising at least one guanylate cyclase activator and at least one nonselectively vasodilating antiobstructive agent.

21. A method of treating partial and global respiratory failure in a patient comprising administering to a patient in need thereof a therapeutically effective amount of a pharmaceutical preparation as claimed in claim 20.

22. A method of treating a disease or disorder in a patient comprising administering to a patient in need thereof a therapeutically effective amount of a pharmaceutical preparation as claimed in claim 20, wherein the disease or disorder is selected from the group consisting of COPD, bronchial asthma, latent pulmonary hypertension, emphysema, combined ventilation disturbances and chronic left heart failure with pulmonary congestion.

23. A pharmaceutical preparation according to claim 20, wherein the guanylate cyclase activator is an active ingredient selected from the group consisting of (9S,11S)-4-amino-2-[1-(2-fluorobenzyl)-1H-pyrazolo[3,4-b]pyridin-3-yl]-6,7,9,10-tetrahydro-5,9-methanopyrimido[4,5-d][1,3,6]oxadiazocin-11-ol, 5-cyclopropyl-2-[1-(2-fluorobenzyl)-1H-pyrazolo[3,4-b]pyridin-3-yl)-pyrimidine-4-amine (BAY-41-2272), 2-[1-(2-fluorobenzyl)-1H-pyrazolo[3,4-b]pyridin-3-yl]-5-(4-morpholinyl)pyrimidine-4,6-diamine (BAY-41-8543), trans-4-acetamidocyclohexyl nitrate (BM-12.1307), 1-[2-(1,3-dimethyl-2-butenylidene)hydrazino]phthalazine (BUDRALAZINE), ethyl 3-{6-[N-(2-hydroxypropyl)-N-ethylamino]-3-pyridazinyl}carbazate (CADRALAZINE), trans-1,4-di(hydroxymethyl)cyclohexane dinitrate ester (CEDO-8956), 1-benzyl-3-[3-(dimethylamino)propoxy]-N-(4-methoxyphenyl)-1H-pyrazole-5-carboxamide (CFM-1571), 4-phenyl-3-(phenylsulfonyl)furazan 2-oxide (CHF-2206), 8-hydroxy-2-(nitratomethyl)-7-nitro-1,4-benzodioxane (E-4701), 1,2,3-propanetriyl trinitrate (GLYCEROL TRINITRATE), 1,4:3,6-dianhydro-D-glucitol dinitrate (ISOSORBIDE DINITRATE), 1,4:3,6-dianhydro-D-glucitol 5-mononitrate (ISOSORBIDE MONONITRATE), 6-methyl-3-[2-(nitrooxy)ethyl]-3,4-dihydro-2H-1,3-benzoxazin-4-one (ITF-1129), 7-[2-[4-(2-chlorophenyl)piperazin-1-yl]ethyl]-1,3-dimethyl-3,7-dihydro-1H-purine-2,6-dione (KMUP-1), N-cyano-N′-(2-nitrooxyethyl)-3-pyridinecarboxamidine (KRN-2391), 3-(2-nitrooxyethyl-3,4-dihydro-2H-1,3-benzoxazin-4-one (SINITRODIL), sodium pentacyanonitrosylferrate(II) (SODIUM NITROPRUSSIDE), N-(3-nitratopivaloyl)cysteine ethyl ester (SPM-3672), 3-[2-(acetamido)propionylthio]-2(R)-[2,2-dimethyl-3-(nitrooxy)propionamido]propionic acid ethyl ester (SPM-5185), 1,4:3,6-dianhydro-2-deoxy-2-[[3-(1,2,3,6-tetrahydro-1,3-dimethyl-2,6-dioxopurin-7-yl)propyl]amino]-L-iditol 5-nitrate (TEOPRANITOL), glutaminyl-glutamyl-aspartyl-cysteinyl-glutamyl-leucyl-cysteinyl-isoleucyl-asparaginyl-valyl-alanyl-cysteinyl-threonyl-glycyl-cysteine (UROGUANYLINE), 1-benzyl-3-[5-(hydroxymethyl)furan-2-yl]-1H-indazole (YC-1), L-arginyl-L-seryl-L-seryl-L-cysteinyl-L-phenylalanylglycylglycyl-L-arginyl-L-methionyl-L-aspartyl-L-arginyl-L-isoleucyl-glycyl- L-alanyl-L-glutaminyl-L-serylglycyl-L-leucylglycyl-L-cysteinyl-L-asparaginyl-L-seryl-L-phenylalanyl-L-arginyl-L-tyrosine, cyclic (4→20) disulfide (ANARITIDE), 1-de-L-leucine-2-de-L-alanine-3-deglycine-4-de-L-proline-5-de-L-arginine-17-L-methionine-atriopeptin-33 (rat) (CARPERITIDE), seryl-prolyl-lysyl-methionyl-valyl-glutaminyl-glycyl-seryl-glycyl-cysteinyl-phenylalanyl-glycyl-arginyl-lysyl-methionyl- aspartyl-arginyl-isoleucyl-seryl-seryl-seryl-seryl-glycyl-leucyl-glycyl-cysteinyl-lysyl-valyl-leucyl-arginyl-arginyl-histidine, cyclic (S-3.10-S-3.26)-disulfide (NESIRITIDE), 4-(N-(4-carboxybutyl)-N-[2-[2-[4-(2-phenylethyl)benzyloxy]phenyl]ethyl]aminomethyl]benzoic acid, 3-[2-(4-chlorophenylsulfanyl)-phenyl]-N-[4-(dimethylamino)butyl]-2-propanamide, 3-[2-(4-chlorophenylsulfanyl)phenyl]-N-[4-(dimethylamino)butyl]-2-propenamide and the pharmacologically acceptable salts of these compounds.

24. A pharmaceutical preparation according to claim 20, wherein the guanylate cyclase activator is an active ingredient selected from the group consisting of BM-12.1307, BUDRALAZINE, CADRALAZINE, GLYCEROL TRINITRATE, ISOSORBIDE DINITRATE, ISOSORBIDE MONONITRATE, KRN-2391, SINITRODIL, SODIUM NITROPRUSSIDE, TEOPRANITOL, ANARITIDE, CARPERITIDE, NESIRITIDE and the pharmacologically acceptable salts of these compounds.

25. A method of treating partial and global respiratory failure in a human in need thereof comprising the step of administering to said human a therapeutically effective amount of a guanylate cyclase activator.

26. A method according to claim 25 wherein the human in need is showing a mismatch of pulmonary ventilation and pulmonary perfusion.

27. The method according to claim 25, wherein the human in need has an exercise-dependent mismatch.

28. The method according to claim 25, wherein the human in need has an age-related mismatch.

29. The method according to claim 25, wherein the human in need has a pathologically caused mismatch.

30. The method according to claim 25, wherein the human in need has a mismatch of V/Q<0.1.

31. The method according to claim 25, wherein the human in need is a COPD patient.

32. The method according to claim 31, wherein the human in need is a COPD patient with a predominant bronchitis component.

33. The method according to claim 31, wherein the human in need is a COPD patient with a V/Q<0.1.

34. The method according to claim 31, wherein the human in need is a COPD patient with an emphysematous component.

35. The method according to claim 31, wherein the human in need is a COPD patient with a V/Q>10.

36. A method according to claim 25, wherein the human in need has orthopnoea.

37. A method according to claim 25, wherein the human in need has sleep apnoea.

38. The method according to claim 25, wherein the human in need has a therapy-induced mismatch.

39. The method according to claim 38, wherein the human in need has a mismatch caused by administration of nonselectively vasodilating medicaments.

40. The method according to claim 39, wherein the nonselectively vasodilating medicament is a non-selectively vasodilating antiobstructive agent.

41. The method according to claim 40, wherein the nonselectively vasodilating antiobstructive agent is selected from the group consisting of endothelin antagonist, Ca channel blocker, ACE inhibitor, ATII antagonist and β blocker.

42. A method of treating muscular dysfunction in a human showing a perfusion/demand mismatch comprising the step of administering to said human a therapeutically effective amount of a guanylate cyclase activator.

43. The method according to claim 25, wherein the guanylate cyclase activator is an active ingredient selected from the group consisting of (9S,11S)-4-amino-2-[1-(2-fluorobenzyl)-1H-pyrazolo[3,4-b]pyridin-3-yl]-6,7,9,10-tetrahydro-5,9-methanopyrimido[4,5-d][1,3,6]oxadiazocin-11-ol, 5-cyclopropyl-2-[1-(2-fluorobenzyl)-1H-pyrazolo[3,4-b]pyridin-3-yl]pyrimidine-4-amine (BAY-41-2272), 2-[1-(2-fluorobenzyl)-1H-pyrazolo[3,4-b]pyridin-3-yl]-5-(4-morpholinyl)pyrimidine-4,6-diamine (BAY-41-8543), trans-4-acetamidocyclohexyl nitrate (BM-12.1307), 1-[2-(1,3-dimethyl-2-butenylidene)hydrazino]-phthalazine (BUDRALAZINE), ethyl 3-{6-[N-(2-hydroxypropyl)-N-ethylamino]-3-pyridazinyl}-carbazate (CADRALAZINE), trans-1,4-di(hydroxymethyl)cyclohexane dinitrate ester (CEDO-8956), 1-benzyl-3-[3(dimethylamino)propoxy]-N-(4-methoxyphenyl)-1H-pyrazole-5-carboxamide (CFM-1571), 4-phenyl-3-(phenylsulfonyl)furazan 2-oxide (CHF-2206), 8-hydroxy-2-(nitratomethyl)-7-nitro-1,4-benzodioxane (E-4701), 1,2,3-propanetriyl trinitrate (GLYCEROL TRINITRATE), 1,4:3,6-dianhydro-D-glucitol dinitrate (ISOSORBIDE DINITRATE), 1,4:3,6-dianhydro-D-glucitol 5-mononitrate (ISOSORBIDE MONONITRATE), 6-methyl-3-[2-(nitrooxy)ethyl]-3,4-dihydro-2H-1,3-benzoxazin-4-one (ITF-1129), 7-[2-[4-(2-chlorophenyl)piperazin-1-yl]ethyl]-1,3-dimethyl-3,7-dihydro-1H-purine-2,6-dione (KMUP-1), N-cyano-N′-(2-nitrooxyethyl)-3-pyridinecarboxamidine (KRN-2391), 3-(2-nitrooxyethyl)-3,4-dihydro-2H-1,3-benzoxazin-4-one (SINITRODIL), sodium pentacyanonitrosyl-ferrate(II) (SODIUM NITROPRUSSIDE), N-(3-nitratopivaloyl)cysteine ethyl ester (SPM-3672), 3-[2-(acetamido)propionylthio]-2(R)-[2,2-dimethyl-3-(nitrooxy)-propionamido]propionic acid ethyl ester (SPM-5185), 1,4:3,6-dianhydro-2-deoxy-2-[[3-(1,2,3,6-tetrahydro-1,3-dimethyl-2,6-dioxopurin-7-yl)propyl]amino]-L-iditol 5-nitrate (TEOPRANITOL), glutaminyl-glutamyl-aspartylcysteinyl-glutamyl-leucyl-cysteinyl-isoleucyl-asparaginyl-valyl-alanyl-cysteinyl-threonyl-glycyl-cysteine (UROGUANYLINE), 1-benzyl-3-[5-(hydroxymethyl)furan-2-yl]-1H-indazole (YC-1), L-arginyl-L-seryl-L-seryl-L-cysteinyl-L-phenylalanylglycylglycyl-L-arginyl-L-methionyl-L-aspartyl-L-arginyl-L-isoleucyl-glycyl- L-alanyl-L-glutaminyl-L-serylglycyl-L-leucylglycyl-L-cysteinyl-Lasparaginyl-L-seryl-L-phenylalanyl-L-arginyl-L-tyrosine, cyclic (4→20) disulfide (ANARITIDE), 1-de-L-leucine-2-de-L-alanine-3-deglycine-4-de-L-proline-5-de-L-arginine-17-L-methionine-atriopeptin-33 (rat) (CARPERITIDE), seryl-prolyl-lysyl-methionyl-valyl-glutaminyl-glycyl-seryl-glycyl-cysteinyl-phenylalanyl-glycyl-arginyl-lysyl-methionyl- aspartyl-arginyl-isoleucyl-seryl-seryl-seryl-seryl-glycyl-leucyl-glycyl-cysteinyl-lysyl-valyl-leucyl-arginyl-arginyl-histidine, cyclic (S-3.10-S-3.26)-disulfide (NESIRITIDE), 4-[N-(4-carboxybutyl)-N-[2-[2-[4-(2-phenylethyl)benzyloxy]phenyl]ethyl]aminomethyl]benzoic acid, 3-[2-(4-chlorophenylsulfanyl)-phenyl]-N-[4-(dimethylamino)butyl]-2-propanamide, 3-[2-(4-chlorophenylsulfanyl)phenyl]-N-[4-(dimethylamino)butyl]-2-propenamide and the pharmacologically acceptable salts of these compounds.

44. The method according to claim 25, wherein the guanylate cyclase activator is an active ingredient selected from the group consisting of BM-12.1307, BUDRALAZINE, CADRALAZINE, GLYCEROL TRINITRATE, ISOSORBIDE DINITRATE, ISOSORBIDE MONONITRATE, KRN-2391, SINITRODIL, SODIUM NITROPRUSSIDE, TEOPRANITOL, ANARITIDE, CARPERITIDE, NESIRITIDE and the pharmacologically acceptable salts of these compounds.

45. The method according to claim 42, wherein the guanylate cyclase activator is an active ingredient selected from the group consisting of (9S,11S)-4-amino-2-[1-(2-fluorobenzyl)-1H-pyrazolo[3,4-b]pyridin-3-yl]-6,7,9,10-tetrahydro-5,9-methanopyrimido[4,5-d][1,3,6]oxadiazocin-11-ol, 5-cyclopropyl-2-[1-(2-fluorobenzyl)-1H-pyrazolo[3,4-b]pyridin-3-yl]pyrimidine-4-amine (BAY-41-2272), 2-[1-(2-fluorobenzyl)-1H-pyrazolo[3,4-b]pyridin-3-yl]-5-(4-morpholinyl)pyrimidine-4,6-diamine (BAY-41-8543), trans-4-acetamidocyclohexyl nitrate (BM-12.1307), 1-[2-(1,3-dimethyl-2-butenylidene)hydrazino]-phthalazine (BUDRALAZINE), ethyl 3-{6-[N-(2-hydroxypropyl)-N-ethylamino]-3-pyridazinyl}-carbazate (CADRALAZINE), trans-1,4-di(hydroxymethyl)cyclohexane dinitrate ester (CEDO-8956), 1-benzyl-3-[3-(dimethylamino)propoxy]-N-(4-methoxyphenyl)-1H-pyrazole-5-carboxamide (CFM-1571), 4-phenyl-3-(phenylsulfonyl)furazan 2-oxide (CHF-2206), 8-hydroxy-2-(nitratomethyl)-7-nitro-1,4-benzodioxane (E-4701), 1,2,3-propanetriyl trinitrate (GLYCEROL TRINITRATE), 1,4:3,6-dianhydro-D-glucitol dinitrate (ISOSORBIDE DINITRATE), 1,4:3,6-dianhydro-D-glucitol 5-mononitrate (ISOSORBIDE MONONITRATE), 6-methyl-3-[2-(nitrooxy)ethyl)-3,4-dihydro-2H-1,3-benzoxazin-4-one (ITF-1129), 7-[2-[4-(2-chlorophenyl)piperazin-1-yl]ethyl]-1,3-dimethyl-3,7-dihydro-1H-purine-2,6-dione (KMUP-1), N-cyano-N′-(2-nitrooxyethyl)-3-pyridinecarbox-amidine (KRN-2391), 3-(2-nitrooxyethyl)-3,4-dihydro-2H-1,3-benzoxazin-4-one (SINITRODIL), sodium pentacyanonitrosyl-ferrate(II) (SODIUM NITROPRUSSIDE), N-(3-nitratopivaloyl)cysteine ethyl ester (SPM-3672), 3-[2-(acetamido)propionylthio]-2(R)-[2,2-dimethyl-3-(nitrooxy)-propionamido]propionic acid ethyl ester (SPM-5185), 1,4:3,6-dianhydro-2-deoxy-2-[[3-(1,2,3,6-tetrahydro-1,3-dimethyl-2,6-dioxopurin-7-yl)propyl]amino]-L-iditol 5-nitrate (TEOPRANITOL), glutaminyl-glutamyl-aspartylcysteinyl-glutamyl-leucyl-cysteinyl-isoleucyl-asparaginyl-valyl-alanyl-cysteinyl-threonyl-glycyl-cysteine (UROGUANYLINE), 1-benzyl-3-[5-(hydroxymethyl)furan-2-yl]-1H-indazole (YC-1), L-arginyl-L-seryl-L-seryl-L-cysteinyl-L-phenylalanylglycylglycyl-L-arginyl-L-methionyl-L-aspartyl-L-arginyl-L-isoleucyl-glycyl-L- alanyl-L-glutaminyl-L-serylglycyl-L-leucylglycyl-L-cysteinyl-Lasparaginyl-L-seryl-L-phenylalanyl-L-arginyl-L-tyrosine, cyclic (4→20) disulfide (ANARITIDE), 1-de-L-leucine-2-de-L-alanine-3-deglycine-4-de-L-proline-5-de-L-arginine-17-L-methionine-atriopeptin-33 (rat) (CARPERITIDE), seryl-prolyl-lysyl-methionyl-valyl-glutaminyl-glycyl-seryl-glycyl-cysteinyl-phenylalanyl-glycyl-arginyl-lysyl-methionyl- aspartyl-arginyl-isoleucyl-seryl-seryl-seryl-seryl-glycyl-leucyl-glycyl-cysteinyl-lysyl-valyl-leucyl-arginyl-arginyl-histidine, cyclic (S-3.10-S-3.26)-disulfide (NESIRITIDE), 4-[N-(4-carboxybutyl)-N-[2-[2-[4-(2-phenylethyl)benzyloxy]phenyl]ethyl]aminomethyl]benzoic acid, 3-[2-(4-chlorophenylsulfanyl)-phenyl]-N-[4-(dimethylamino)butyl]-2-propanamide, 3-[2-(4-chlorophenylsulfanyl)phenyl]-N-[4-(dimethylamino)butyl]-2-propenamide and the pharmacologically acceptable salts of these compounds.

46. The method according to claim 42, wherein the guanylate cyclase activator is an active ingredient selected from the group consisting of BM-12.1307, BUDRALAZINE, CADRALAZINE, GLYCEROL TRINITRATE, ISOSORBIDE DINITRATE, ISOSORBIDE MONONITRATE, KRN-2391, SINITRODIL, SODIUM NITROPRUSSIDE, TEOPRANITOL, ANARITIDE, CARPERITIDE, NESIRITIDE and the pharmacologically acceptable salts of these compounds.
